(19) United States
(12) Patent Application Publication
(10) Pub. No.: US 2024/0342196 A1
(43) Pub. Date: Oct. 17, 2024

(54) TARGETING SELECT LIPID SIGNALING TO COUNTER TYPE 1 DIABETES (T1D)

(71) Applicants: The UAB Research Foundation, Birmingham, AL (US); The Regents of the University of California, Oakland, CA (US)

(72) Inventors: Sansanka Ramanadham, Birmingham, AL (US); Tayleur D. White, Birmingham, AL (US); Bruce Hammock, Davis, CA (US); Sung Hee Hwang, Davis, CA (US)

(21) Appl. No.: 18/634,143

(22) Filed: Apr. 12, 2024

Related U.S. Application Data
(60) Provisional application No. 63/495,980, filed on Apr. 13, 2023.

(51) Int. Cl. A61K 31/64; A61K 31/202; A61K 31/4468; A61P 3/10

(52) U.S. Cl. CPC A61K 31/64; A61K 31/202; A61K 31/4468; A61P 3/10

(57) ABSTRACT

Disclosed herein are lipid signaling pathways that can be targeted to prevent or delay the onset of type 1 diabetes (T1D). In particular, disclosed herein a pathway that can be modulated by a chemical inhibitor. Utilizing this inhibitor, evidence was generated suggesting that it is beneficial in reducing the incidence of T1D.

### CROSS-REFERENCE TO RELATED APPLICATIONS

This application claims benefit of U.S. Provisional Application No. 63/495,980, filed Apr. 13, 2023, which is hereby incorporated herein by reference in its entirety.

### STATEMENT OF GOVERNMENT INTEREST

This invention was made with Government Support under Grant Nos. ES030443 and ES004699 awarded by the National Institutes of Health. The Government has certain rights in the invention.

### BACKGROUND

Type 1 diabetes (T1D) is a consequence of autoimmune destruction of pancreatic islet β-cells, but the involved processes remain unclear.

### SUMMARY

Disclosed herein are lipid signaling pathways that can be targeted to prevent or delay the onset of type 1 diabetes (T1D). In particular, disclosed herein a pathway that can be modulated by a chemical inhibitor. Utilizing this inhibitor, evidence was generated suggesting that it is beneficial in reducing the incidence of T1D.

We demonstrated that the Ca²⁺-independent phospholipase A2β (iPLA2β) is induced under a diabetic milieu and participates in β-cell death. The iPLA2β hydrolyzes membrane phospholipids at the sn-2 substituent to release a fatty acid (i.e., arachidonic acid), that is metabolized to generate proinflammatory (pi) lipids. We find that global reduction of iPLA2β reduces T1D incidence. Given the importance of T-lymphocytes in T1D development, we investigated the contribution of iPLA2β-derived lipids (iDLs) from T-lymphocytes with adoptive transfer and ex vitro functional approaches. The CD4₊/8₊ T-cells were purified from pre-diabetic female NOD and NOD.iPLA2β^{+/-} (HET) spleens. Different combinations of the cells (NOD4₊8, NOD4₊HET8, and NOD8₊HET4) were then administered (i.p. 7.5×10⁶: 2.5×10⁶) to 4-week-old female immunodeficient NOD.scid mice. We find that scid recipients of a combination that included HET4 or HET8 had higher plasma insulin, decreased insulitis, and increased B-cell mass, compared to recipients of NOD4₊NOD8. Intriguingly, T1D incidences in the NOD4₊HET8 and NOD8₊HET4 were significantly reduced, relative to NOD4₊NOD8 recipients. Surprisingly, the T-cell phenotype was similar in all recipient groups. However, lipidomics analyses revealed a lower abundance of pi eicosanoids (i.e., PGs, DHETs) in the non-diabetic NOD.scid mice, compared to diabetic counterparts, suggesting that downstream signaling of iDLs contributes to T1D onset. In fact, targeting PGE₂ and DHET signaling with grapiprant (EP4 receptor antagonist) and EC5026 (soluble epoxide hydrolase inhibitor) significantly reduced IFN-γ production by Th1 helper cells in the NOD. These findings suggest the importance of iPLA2β activation in T-cells in T1D development and that targeting this enzyme can be beneficial in delaying or preventing T1D onset.

The details of one or more embodiments of the invention are set forth in the accompanying drawings and the description below. Other features, objects, and advantages of the invention will be apparent from the description and drawings, and from the claims.

### BRIEF DESCRIPTION OF FIGURES

FIG. 1 shows T1D incidence after treatment with vehicle, EC5026 (EC), Grapiprant (GP), or a combination thereof.

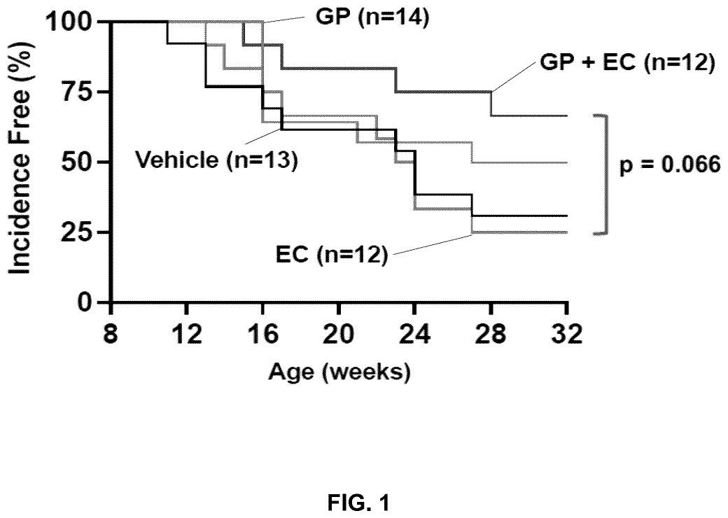

FIG. 2 shows histological assessment of tissues from NOD mice treated with Grapirant±EC5026. Female NOD mice were treated as above (KB-referring to incidence analyses). When the mice became diabetic or were diabetes-free at the end of the study (>30 weeks of age), they were killed and the kidneys, heart, and liver tissues were excised. The tissues were embedded paraffin and processed for histological assessments. The sections were examined a certified Anatomic Pathologist in a blinded fashion and then imaged. The images presented are representative (n=3) of tissues from animals that were treated with vehicle alone, grapiprant alone, EC5026 alone, or combination of grapiprant and EC5026 that turned diabetic or remained diabetes-free by the indicated age in weeks.

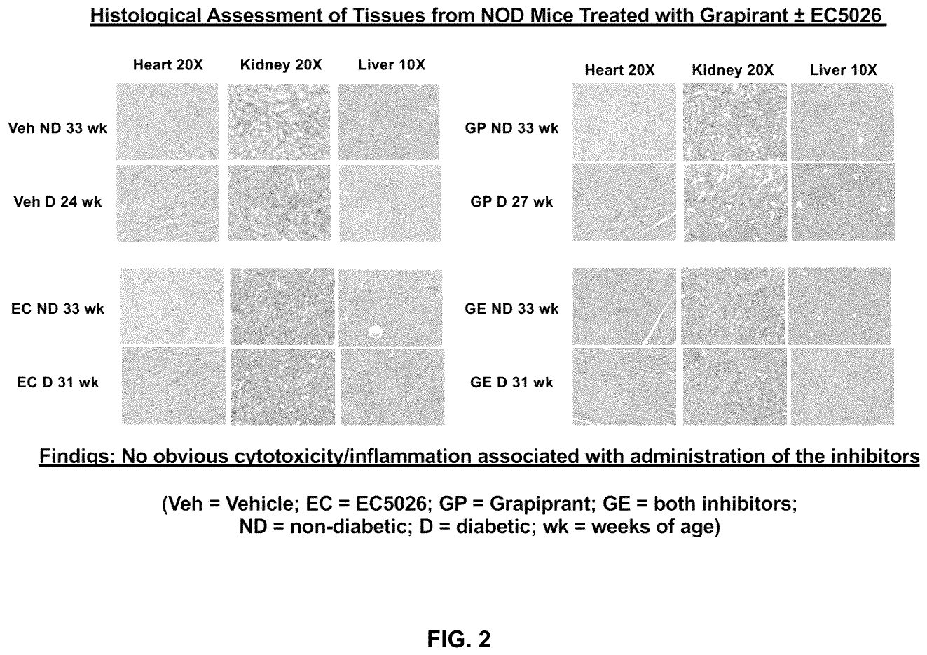

FIG. 3 shows insuline measurements in plasma of NOD mice treated with Grapirant±EC5026. Female NOD mice were treated as above (KB-referring to incidence analyses). When the mice became diabetic or were diabetes-free at the end of the study (>30 weeks of age), they were killed and the blood collected for insulin analyses by ELISA. The data presented are mean±SEM of insulin levels in the plasma of mice treated with vehicle alone, grapiprant alone, EC5026 alone, or combination of grapiprant and EC5026 that turned diabetic or remained diabetes-free.

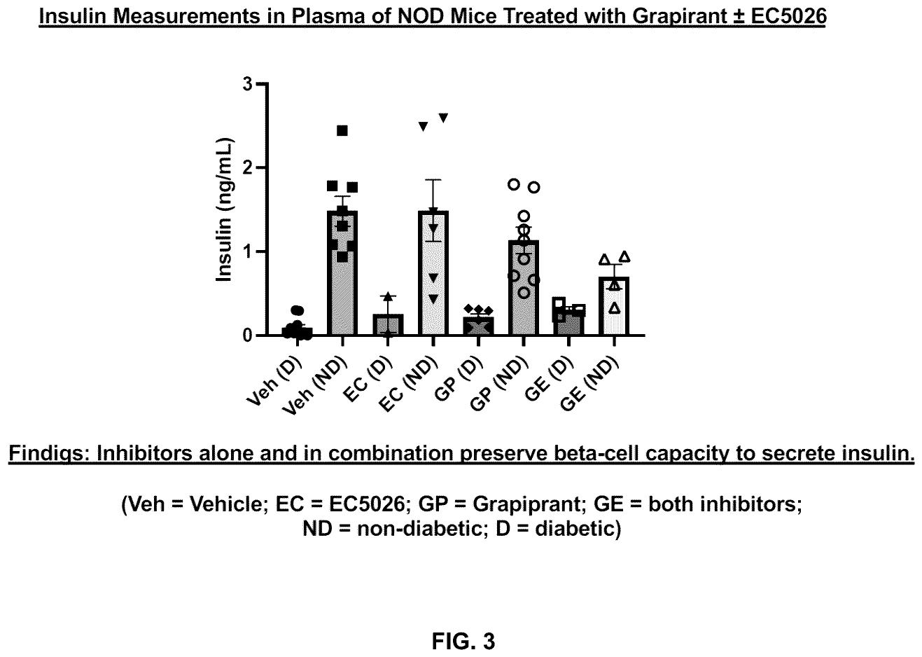

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

### Definitions

It must be noted that, as used in the specification and the appended claims, the singular forms “a,” “an,” and “the” include plural referents unless the context clearly dictates otherwise.

“Epoxide hydrolases” (“EH;” EC 3.3.2.3) are enzymes in the alpha beta hydrolase fold family that add water to 3-membered cyclic ethers termed epoxides.

“Soluble epoxide hydrolase” (“sEH”) is an epoxide hydrolase which in endothelial and smooth muscle cells converts EETs to dihydroxy derivatives called dihydroxyeicosatrienoic acids (“DHETs”). The cloning and sequence of the murine sEH is set forth in Grant et al., J. Biol. Chem. 268 (23): 17628-17633 (1993). The cloning, sequence, and accession numbers of the human sEH sequence are set forth in Beetham et al., Arch. Biochem. Biophys. 305 (l): 197-201 (1993). The evolution and nomenclature of the gene is discussed in Beetham et al., DNA Cell Biol. 14 (1): 61-71 (1995). Soluble epoxide hydrolase represents a single highly conserved gene product with over 90% homology between rodent and human (Arand et al., FEBS Lett., 338:251-256 (1994)). Unless otherwise specified, as used herein, the terms “soluble epoxide hydrolase” and “sEH” refer to human sEH.

Unless otherwise specified, as used herein, the term “sEH inhibitor” (also abbreviated as “sEHI”) refers to an inhibitor of human sEH. Preferably, the inhibitor does not also inhibit the activity of microsomal epoxide hydrolase by more than 25% at concentrations at which the inhibitor inhibits sEH by at least 50%, and more preferably docs not inhibit mEH by more than 10% at that concentration. For convenience of reference, unless otherwise required by context, the term “sEH inhibitor” as used herein encompasses prodrugs which are metabolized to active inhibitors of sEH. Further for convenience of reference, and except as otherwise required by context, reference herein to a compound as an inhibitor of sEH includes reference to derivatives of that compound (such as an ester of that compound) that retain activity as an sEH inhibitor.

The term “therapeutically effective amount” refers to that amount of the compound being administered sufficient to prevent, mitigate, decrease, reverse the development of one or more of the symptoms of the disease, condition or disorder being treated.

The terms “sustained release” and “extended release” are used in their conventional sense to refer to a drug formulation that provides for gradual release of a drug over an extended period of time, for example, 12 hours or more, and that preferably, although not necessarily, results in substantially steady-state blood levels of a drug over an extended time period.

The terms “systemic administration” and “systemically administered” refer to a method of administering agent to a mammal so that the agent/cells is delivered to sites in the body, including the targeted site of pharmaceutical action, via the circulatory system. Systemic administration includes, but is not limited to, oral, intranasal, rectal and parenteral (z.e., other than through the alimentary tract, such as intramuscular, intravenous, intra-arterial, transdermal and subcutaneous) administration.

The phrase “cause to be administered” refers to the actions taken by a medical professional (e.g., a physician), or a person controlling medical care of a subject, that control and/or permit the administration of the agent(s)/compound(s)/cell(s) at issue to the subject. Causing to be administered can involve diagnosis and/or determination of an appropriate therapeutic or prophylactic regimen, and/or prescribing particular agent(s)/compounds/cell(s) for a subject. Such prescribing can include, for example, drafting a prescription form, annotating a medical record, and the like.

The terms “patient,” “subject” or “individual” interchangeably refers to a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster).

As used herein, the term “an agent that inhibits EP4 activity” or “an EP4 inhibitor” refers to an agent that reduces or attenuates the biological activity of an EP4 receptor. Such agents may include proteins such as anti-EP4 antibodies, nucleic acids, amino acids, peptides carbohydrates, small molecules (organic or inorganic), or any other compound or composition which decreases the activity of an EP4 receptor either by reducing the amount of EP4 receptor present in a cell, or by decreasing the binding or signaling activity of the EP4 receptor.

As used herein, the term “EP4 receptor activity” or “EP4 activity” refers to an EP4-mediated increase in cAMP levels upon PGE2 stimulation.

As used herein, the term “a selective EP4 inhibitor” is an agent that inhibits EP4 activity with an IC50 at least 10-fold less, preferably, at least 100-fold less than the IC50 for inhibition of EP1, EP2, or EP3 activity, as determined by standard methods known in the art.

As used herein, the term “diabetes” or “diabetes mellitus” refers to a disease or condition is which a subject has high blood sugar. Examples of diabetes that may be treated with a compound, pharmaceutical composition, or method described herein include type I diabetes (type I diabetes mellitus), which is characterized by the subject's failure to produce insulin or failure to produce sufficient insulin for the subject's metabolic needs; type II diabetes (type II diabetes mellitus), which is characterized by insulin resistance (i.e. the failure of the subject (e.g. subject's cells) to use insulin properly; and gestational diabetes, which is high blood sugar during pregnancy. In embodiments, diabetes is type I diabetes. In embodiments, diabetes is type II diabetes. In embodiments, diabetes is gestational diabetes. In embodiments, diabetes is a disease or condition in which a subject has high blood sugar as determined by an A1C test (e.g. 6.5% or greater), fasting plasma glucose test (e.g. 126 mg/dL or greater), or oral glucose tolerance test (e.g. 200 mg/dL or greater). In embodiments, the diabetes is associated with Wolfram Syndrome.

Soluble Epoxide Hydrolase (sEH) Inhibitor

Inhibitors of sEH have been previously disclosed. Each of these compounds are intended to be within the scope of the current disclosure.

sEH inhibitors are described in U.S. Pat. No. 11,873,288 and Publication No. US2021/10161881, Publication No. US2017/0174665, and Publication No. CN113264922A, which are incorporated by reference in their entireties for the teaching of these inhibitors and their uses. In some embodiments, the sEH inhibitor is a compound of Formula I (EC5026):

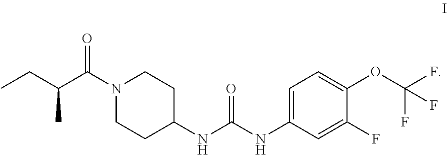 [SMILES: CC[C@H](C)C(=O)N1CCC(NC(=O)Nc2ccc(OC(F)(F)F)c(F)c2)CC1]

The disclosed compounds may exist as salts. The present invention includes such salts. Typically, the salts used are pharmaceutically acceptable salts. Pharmaceutically acceptable salts include salts of the active compounds which are prepared with relatively nontoxic acids or bases, depending on the particular substituents found on the compounds described herein. When compounds of Formula II contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of Formula II contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of Formula II contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds can exist in unsolvated forms as well as solvated forms, including hydrated forms. In general, the solvated forms are equivalent to unsolvated forms and are encompassed within the scope of the present invention. Certain compounds may exist in multiple crystalline or amorphous forms. In general, all physical forms are equivalent for the uses contemplated by the present invention and are intended to be within the scope of the present invention.

Certain compounds possess asymmetric carbon atoms (optical centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques.

Isomers include compounds having the same number and kind of atoms, and hence the same molecular weight, but differing in respect to the structural arrangement or configuration of the atoms.

It will be apparent to one skilled in the art that certain compounds of this invention may exist in tautomeric forms, all such tautomeric forms of the compounds being within the scope of the invention. Tautomer includes one of two or more structural isomers which exist in equilibrium and which are readily converted from one isomeric form to another.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, the compounds may also contain unnatural proportions of atomic isotopes at one or more of the atoms that constitute such compounds. For example, the compounds of Formula II maybe radiolabeled with radioactive isotopes, such as for example deuterium (²H), tritium (³H), iodine-125 (¹²⁵I), carbon-13 (¹³C), or carbon-14 (¹⁴C). All isotopic variations of the compounds of Formula II, whether radioactive or not, are encompassed within the scope of the present invention.

In addition to salt forms, the compounds can be prepared as prodrugs. Prodrugs of the compounds described herein are those compounds that readily undergo chemical changes under physiological conditions to provide the compounds of Formula I. Additionally, prodrugs can be converted to the compounds of Formula I by chemical or biochemical methods in an ex vivo environment. For example, prodrugs can be slowly converted to the compounds of Formula II when placed in a transdermal patch reservoir with a suitable enzyme or chemical reagent.

Other inhibitors of the soluble epoxide hydrolase which have Ki's of less than 500 nmolar and pharmacokinetic properties that result in increased levels of long chain epoxyfatty acids leading to resolution of inflammation, pain and cell senescence.

### EP4 Receptor Inhibitor

EP4 receptor antagonists are described in U.S. Pat. No. 10,973,834, which is incorporated by reference in its entirety for the teaching of these compounds, methods of making, and administering. In certain embodiments, the EP4 receptor inhibitor (also know as grapiprant) has the formula:

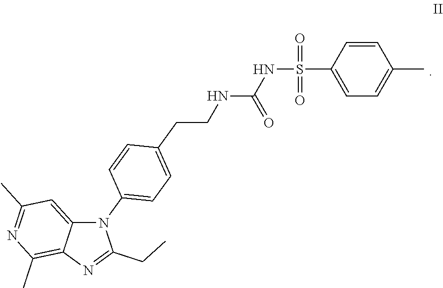

In some embodiment, grapiprant is in crystal form. In some embodiments, grapiprant is in polymorph Form A, as described in U.S. Pat. Nos. 7,960,407 and 9,265,756, the contents of which are incorporated herein by reference in their entireties. In some embodiments, polymorph Form A of grapiprant is characterised by a powder X-ray diffraction pattern obtained by irradiation with Cu Kα radiation which includes main peaks at 2-Theta° 9.8, 13.2, 13.4, 13.7, 14.1, 17.5, 19.0, 21.6, 24.0 and 25.7+/−0.2. In some embodiments, polymorph Form A of compound II is characterised by differential scanning calorimetry (DSC) in which it exhibits an endothermic event at about 160° C. In some embodiments, polymorph Form A of grapiprant exhibits an X-ray powder diffraction pattern having characteristic peaks expressed in degrees 2-theta at about 9.9, about 13.5, about 14.3, about 16.1, about 17.7, about 21.8, about 24.14, and about 25.8. In some embodiments, polymorph Form A of grapiprant exhibits a differential scanning calorimetry profile having showed an endotherm/exotherm at about 155-170° C. In some embodiments, polymorph Form A of grapiprant exhibits a thermogravimetric analysis showing a loss of mass of 0.5-0.6% when heated from about 300 to about 150° C.

In some embodiments, the EP4 receptor inhibitor is L-161,982, having the formula:

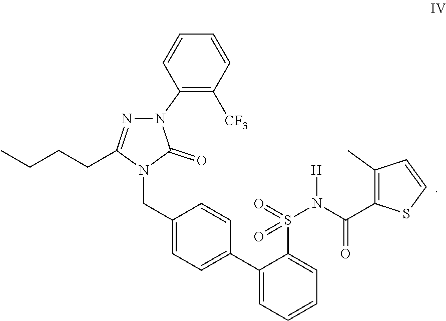 [SMILES: CCCCc1nn(-c2ccccc2C(F)(F)F)c(=O)n1Cc1ccc(-c2ccccc2S(=O)(=O)NC(=O)c2sccc2C)cc1]

L-161,982 is a potent and selective EP4 receptor antagonist. It demonstrates selective binding to human EP4 receptors with a Ki value of 0.024 μM compared to other receptors of the prostanoid family, EP1, EP2, EP3, DP, FP, and IP, with Ki values of 17, 23, 1.9, 5.1, 5.6, and 6.7 μM, respectively. L-161,982 at 10 mg/kg/day suppresses PGE2-stimulated bone formation in young rats and at 100 nM reverses the anti-inflammatory action of PGE2 in LPS-activated human macrophages. At 10 μM L-161982 blocks PGE2-induced cell proliferation in HCA-7 colon cancer cells.

In some embodiments, the EP4 receptor inhibitor is an indole or indoline cyclopropyl amide derivative described in U.S. Pat. No. 8,158,671, which is incorporated by reference in its entirety for the teaching of these compounds and their uses. For example, in some embodiments, the EP4 receptor inhibitor is MF-766, which has the formula:

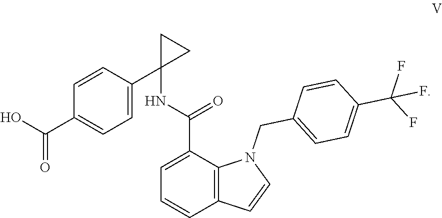 [SMILES: O=C(O)c1ccc(C2(NC(=O)c3cccc4ccn(Cc5ccc(C(F)(F)F)cc5)c34)CC2)cc1]

MF-766 is a highly potent, selective and orally active EP4 antagonist with a Ki of 0.23 nM. MF-766 behaves as a full antagonist with an IC₅₀ of 1.4 nM (shifted to 1.8 nM in the presence of 10% HS) in the functional assay. MF-766 can be used for cancer and inflammation diseases research. MF-766 (0.01-10 μM; pretreatment for 1 h and then stimulated with 50 ng/ml IL-2; with and without 0.33 μM PGE2; 18 hours) reverses PGE2-suppressed IFN-γ secretion in human NK cells. Additionally, NK cell viability is not affected by MF-766.

In some embodiments, the EP4 receptor inhibitor is EP4 Receptor Antagonist 1, having the formula:

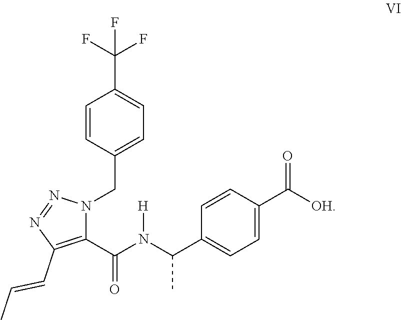 [SMILES: C/C=C/c1nnn(Cc2ccc(C(F)(F)F)cc2)c1C(=O)N[C@@H](C)c1ccc(C(=O)O)cc1]

EP4 receptor antagonist 1 is an antagonist of the prostaglandin E2 (PGE₂) receptor EP4 that has an IC₅₀ value of 6.1 nM in a calcium flux assay using CHO cells co-expressing the human receptor and Gα16. It is selective for EP4 over EP1, EP2, and EP3 receptors (IC₅₀s=>10,000 nM for all). EP4 receptor antagonist 1 inhibits PGE2-induced β-arrestin recruitment in HEK293 cells expressing EP4. It reverses ERK phosphorylation induced by PGE₂ in CHO cells expressing EP4 and decreases GM-CSF-induced expression of II1b, II4ra, II6, Arg1, Cox2, and II10 in RAW 264.7 cells when used at a concentration of 10 μM.

### Resolvin

In some embodiments, the method further involves administering to the subject a composition comprising a resolvin. Resolvins are described in U.S. Pat. No. 8,933,270, which is incorporated by reference in its entirety for the teaching of these compounds and their uses. Resolvins are a family of potent lipid mediators derived from both eicosapentaenoic acid and docosahexaenoic acid. In addition to being anti-inflammatory, resolvins promote the resolution of the inflammatory response back to a non-inflamed state. In some embodiments, the resolvin is Resolvin D1 having the formula:

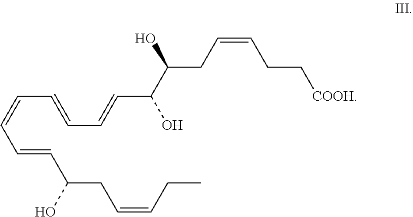 [SMILES: CC/C=C\C[C@H](O)/C=C/C=C\C=C\C=C\[C@@H](O)[C@@H](O)C/C=C\CCC(=O)O]

Resolvin D1 (RvD1) is produced physiologically from the sequential oxygenation of DHA by 15- and 5-lipoxygenase. A 17 (R)-epimer of RvD1 can also be generated in aspirin-treated samples. Both RvD1 and its 17 (R)-configuration reduce human polymorphonuclear leukocyte (PMNL) transendothelial migration, the earliest event in acute inflammation, with EC₅₀ values of ˜30 nM. RvD1 and its aspirin-triggered form also exhibit a dose-dependent reduction in leukocyte infiltration in a mouse model of peritonitis with a maximal inhibition of ˜35% at a 10-100 ng dose.

### Pharmaceutical Compositions and Methods

In another aspect is provided a pharmaceutical composition including a pharmaceutically acceptable excipient and a compound, or pharmaceutically acceptable salt thereof, as described herein. The compounds and methods of this invention may be used for treating patients with diabetes.

The compounds of the present disclosure may be administered by a variety of methods, e.g., orally or by injection (e.g. subcutaneous, intravenous, intraperitoneal, etc.). Depending on the route of administration, the active compounds may be coated in a material to protect the compound from the action of acids and other natural conditions which may inactivate the compound. They may also be administered by continuous perfusion/infusion of a disease or wound site.

To administer the therapeutic compound by other than parenteral administration, it may be necessary to coat the compound with, or co-administer the compound with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The therapeutic compound may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the therapeutic compound in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The therapeutic compound can be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation.

Active compounds are administered at a therapeutically effective dosage sufficient to treat a condition associated with a condition in a patient. A “therapeutically effective amount” preferably reduces the amount of symptoms of the condition in the infected patient by at least about 20%, more preferably by at least about 40%, even more preferably by at least about 60%, and still more preferably by at least about 80% relative to untreated subjects. For example, the efficacy of a compound can be evaluated in an animal model system that may be predictive of efficacy in treating the disease in humans, such as the model systems shown in the examples and drawings.

The actual dosage amount of a compound of the present disclosure or composition comprising a compound of the present disclosure administered to a subject may be determined by physical and physiological factors such as age, sex, body weight, severity of condition, the type of disease being treated, previous or concurrent therapeutic interventions, idiopathy of the subject and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

An effective amount typically will vary from about 0.001 mg/kg to about 1,000 mg/kg, from about 0.01 mg/kg to about 750 mg/kg, from about 100 mg/kg to about 500 mg/kg, from about 1.0 mg/kg to about 250 mg/kg, from about 10.0 mg/kg to about 150 mg/kg in one or more dose administrations daily, for one or several days (depending, of course, of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some particular embodiments, the amount is less than 10,000 mg per day with a range, for example, of 750 mg to 9,000 mg per day.

The effective amount may be less than 1 mg/kg/day, less than 500 mg/kg/day, less than 250 mg/kg/day, less than 100 mg/kg/day, less than 50 mg/kg/day, less than 25 mg/kg/day or less than 10 mg/kg/day. It may alternatively be in the range of 1 mg/kg/day to 200 mg/kg/day. For example, regarding treatment of diabetic patients, the unit dosage may be an amount that reduces blood glucose by at least 40% as compared to an untreated subject. In another embodiment, the unit dosage is an amount that reduces blood glucose to a level that is ±10% of the blood glucose level of a non-diabetic subject.

In other non-limiting examples, a dose may also comprise from about 1 microgram/kg/body weight, about 5 microgram/kg/body weight, about 10 micro-gram/kg/body weight, about 50 microgram/kg/body weight, about 100 micro-gram/kg/body weight, about 200 microgram/kg/body weight, about 350 micro-gram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milli-gram/kg/body weight, about 350 milligram/kg/body weight, about 500 milligram/kg/body weight, to about 1,000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In certain embodiments, a pharmaceutical composition of the present disclosure may comprise, for example, at least about 0.1% of a compound of the present disclosure. In other embodiments, the compound of the present disclosure may comprise between about 2% to about 75% of the weight of the unit, or between about 25% to about 60%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12 hour intervals. In some embodiments, the agent is administered once a day.

The agent(s) may be administered on a routine schedule. As used herein a routine schedule refers to a predetermined designated period of time. The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may taken orally and that the timing of which is or is not dependent upon food intake. Thus, for example, the agent can be taken every morning and/or every evening, regardless of when the subject has eaten or will eat.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

### Background

EC5026 (EC) is an inhibitor of soluble epoxide hydrolase, which preserves inflammation resolving lipid epoxides and generates often proiflammatory lipid diols. Grapiprant (GP) is an inhibitor of the EP4 receptor which transduces signaling of PGE2, an inflammatory lipid. We find both PGE2 and DHETs and elevated in the prediabetic NOD mouse, an autoimmune model of T1D. Increases in PGE2 occur earlier than DHETs.

### Protocol

To determine the impact of their signaling on T1D incidence, we treated NOD mice with either GP or EC alone starting at 4 weeks of age or in combination, where GP was started at 4 weeks of age and EC was introduced at 10 weeks of age. T1D incidence was recorded when blood glucose levels ≥275 mg/ml were measured in two consecutive weeks.

### Results

Over a 30-week monitoring period, NOD treated with vehicle only reached 75% T1D incidence. EC alone was without effect and GP was somewhat effective in reducing T1D incidence to 50%. However, addition of EC produced a greater reduction in T1D (ca. 30%) that fell short of significance.

These findings suggest that EC inclusion can delay T1D onset and reducing T1D incidence. This is the first demonstration of EC5026 having beneficial effects in ameliorating T1D and has motivated us to go forward with this IP application.

### Example 2

Type 1 diabetes (T1D) is a consequence of autoimmune destruction of pancreatic islet β-cells, but the involved processes remain unclear. We demonstrated that the Ca²⁺-independent phospholipase A₂β (iPLA₂β) is induced under a diabetic milieu and participates in β-cell death. The iPLA₂β hydrolyzes membrane phospholipids at the sn-2 substituent to release a fatty acid (i.e., arachidonic acid), that is metabolized to generate proinflammatory (pi) lipids. We find that global reduction of iPLA₂β reduces T1D incidence. Given the importance of T-lymphocytes in T1D development, we investigated the contribution of iPLA₂β-derived lipids (iDLs) from T-lymphocytes with adoptive transfer and ex vitro functional approaches. The CD4⁺/8⁺ T-cells were purified from pre-diabetic female NOD and NOD.iPLA₂β^{+/-} (HET) spleens. Different combinations of the cells (NOD4₊8, NOD4₊HET8, and NOD8₊HET4) were then administered (i.p. 7.5×10⁶: 2.5×10⁶) to 4-week-old female immunodeficient NOD.scid mice. We find that scid recipients of a combination that included HET4 or HET8 had higher plasma insulin, decreased insulitis, and increased β-cell mass, compared to recipients of NOD4₊NOD8. Intriguingly, T1D incidences in the NOD4₊HET8 and NOD8₊HET4 were significantly reduced, relative to NOD4₊NOD8 recipients. Surprisingly, the T-cell phenotype was similar in all recipient groups. However, lipidomics analyses revealed a lower abundance of pi eicosanoids (i.e., PGs, DHETs) in the non-diabetic NOD.scid mice, compared to diabetic counterparts, suggesting that downstream signaling of iDLs contributes to T1D onset. In fact, targeting PGE₂ and DHET signaling with grapiprant (EP4 receptor antagonist) and EC5026 (soluble epoxide hydrolase inhibitor) significantly reduced IFN-γ production by Th1 helper cells in the NOD. These findings suggest the importance of iPLA₂β activation in T-cells in T1D development and that targeting this enzyme can be beneficial in delaying or preventing T1D onset.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A method for treating type 1 diabetes (T1D) in a subject, the method comprising administering to the subject a therapeutically effective amount of a soluble epoxide hydrolase (sEH) inhibitor, an EP4 receptor inhibitor, or a combination thereof.

2. The method of claim 1, wherein the sEH inhibitor is EC5026.

3. The method of claim 2, wherein EC5026 is administered

4. The method of claim 1, wherein the EP4 receptor inhibitor is Grapiprant.

5. The method of claim 3, wherein Grapiprant is administered at a dosage of from 0.01 to 0.10 mg/ml in acid drinking water.

6. The method of claim 4, wherein Grapiprant is administered at a dosage of 0.07 mg/ml in acid drinking water.

7. The method of claim 1, wherein the EP4 receptor inhibitor is L-161,982, MF-766, or EP4 Receptor Antagonist 1.

8. The method of claim 1, further comprising administering to the subject a composition comprising a resolvin.

9. The method of claim 7, wherein the resolvin is Resolvin D1.

10. The method of claim 1, wherein the subject has been diagnosed with autoantibody positivity.
